(19) United States
(12) Patent Application Publication
(10) Pub. No.: US 2024/0075206 A1
(43) Pub. Date: Mar. 7, 2024

(54) ENTERAL FEEDING SYRINGE INSTALLATION AND DETECTION

(71) Applicant: KPR U.S., LLC, Mansfield, MA (US)

(72) Inventors: Samuel ALEXANDER, St. Louis, MO (US); Laura Brames, Chesterfield, MO (US); Kenneth Michael Breitweiser, Brighton, IL (US); Emma Buckles, St. Louis, MO (US); Pang Kian Chan, Singapore (SG); Meghan Fox, St. Louis, MO (US); Chaw Pheng Koh, Singapore (SG); Choon Kwang Yeo, Singapore (SG); Georgia Burlingame, St. Louis, MO (US)

(21) Appl. No.: 18/460,190

(22) Filed: Sep. 1, 2023

Related U.S. Application Data
(60) Provisional application No. 63/374,433, filed on Sep. 2, 2022.

(51) Int. Cl. A61M 5/168; A61M 5/142

(52) U.S. Cl. CPC A61M 5/16804; A61M 5/14228; A61M 2205/14; A61M 2205/3317

(57) ABSTRACT

A device and method of controlling a device. The device may include a controller for controlling operation of a pumping device to producing a flow of fluid in the pump set, the controller including a processor and a memory, the controller being adapted to store in the memory, syringe size data, the controller configured to execute in the processor a syringe detection program to determine a presence and size of the syringe loaded onto a flow control apparatus.

### CROSS REFERENCE TO RELATED APPLICATION

This application claims priority to U.S. Provisional Application No. 63/374,433 filed on Sep. 2, 2022 and titled: “ENTERAL FEEDING SYRINGE DETECTION,” the entirety of which is incorporated by reference herein.

### FIELD

The present disclosure generally relates to fluid delivery, and more particularly to an enteral feeding pump assembly configured to detect a syringe loaded on the assembly.

### BACKGROUND

Administering medicine or nutrition to a patient who cannot intake the medicine or nutrition orally can be affected by utilizing peristaltic flow control systems. Typically, in such systems, fluid is delivered to the patient by a pump set including a flexible elastomeric tubing loaded on a flow control apparatus, such as a peristaltic pump, which delivers fluid to the patient at a controlled rate of delivery. The peristaltic pump usually has a housing that includes a rotor operatively engaged to a motor through a gearbox. The rotor drives fluid through the flexible tubing of the pump set by the peristaltic action effected by reversible compression created by impingement, e.g., pinching, by one or more rollers on the rotor. Rotation of the rotor progressively compresses the elastomeric tubing that drives the fluid at a controlled rate. The pump set may have a valve mechanism for permitting or preventing fluid flow communication through the pump set. The flow control system may also have a controller that operatively regulates the one or more motors which effectively controls fluid flow.

Peristaltic pumps operate by delivering fluid in small charges called “aliquots”. The rotor engages elastomeric tubing of the pump set, pinching off a portion of the elastomeric tubing and pushing fluid forward of the pinch point, e.g., closer to the patient than to the source of fluid toward the patient. Typically, the volume of fluid to be administered to the patient is controlled in the pump by counting the number of aliquots, each being of substantially the same volume, and stopping when the number reaches an amount corresponding to the total desired volume of fluid to be delivered. Peristaltic pumps are sanitary and generally accurate and therefore very useful in the administration of medication and therapeutic fluids to the patient.

### BRIEF DESCRIPTION OF THE DRAWINGS

FIG. 1 is a perspective view of a feeding system including an enteral feeding pump, a pump support, a feeding set assembly and a syringe.

FIG. 2 is a fragmentary, perspective view of the feeding system including the enteral feeding pump, and part of the feeding set assembly.

FIG. 3 is the perspective view of FIG. 2, but with portions of a cassette of the feeding set assembly removed.

FIG. 4 is a front perspective view of the enteral feeding pump.

FIG. 5 is a front perspective view of a pump support and syringe of the feeding set assembly.

FIG. 6 is a front perspective view of the pump support.

FIG. 7A is a front perspective view of a syringe holder of the pump support.

FIG. 7B is a partial view of aspects of FIG. 7A.

FIG. 7C is a partial view of aspects of FIG. 7A.

FIG. 8 is a block diagram showing components of the enteral feeding pump that may be utilized to implement one or more aspects disclosed herein.

FIG. 9 is a flow chart of a syringe size detection routine.

FIGS. 10A and 10B show one example of a flange holder according to aspects of the disclosure.

FIG. 11 show an example of a flange holder according to aspects of the disclosure.

FIG. 12 show an example of a flange holder according to aspects of the disclosure.

FIG. 13 show an example of a flange holder according to aspects of the disclosure.

FIG. 14 show an example of a flange holder according to aspects of the disclosure.

FIG. 15 shows an example of a flange holder according to aspects of the disclosure.

FIG. 16 shows an example of a flange holder according to aspects of the disclosure.

FIGS. 17A and 17B show examples of followers according to aspects of the disclosure.

FIG. 18 shows an example of a follower according to aspects of the disclosure.

FIG. 19 shows an example of a follower according to aspects of the disclosure.

FIGS. 20 and 21 show examples of followers according to aspects of the disclosure.

FIG. 22 shows an example of a follower according to aspects of the disclosure.

FIG. 23 shows an example of a follower according to aspects of the disclosure.

FIGS. 24A and 24B show a top view and a partial see-through bottom view of an example of a follower according to aspects of the disclosure.

### DETAILED DESCRIPTION

One or more aspects of the present disclosure pertain to enteral feeding pumps configured to mount and detect a syringe assembly for delivering enteral feeding fluid to a patient (e.g., infant). Any one or more advantageous features or structures that provide or facilitate any one or more of such features may be implemented in an enteral feeding pump employed in various commercial and industrial applications. Thus, although the detailed discussion is directed to an enteral feeding pump with a feeding set assembly including a cassette, any one or more features of the disclosure may be embodied or implemented in other pumps. For example, although the example of a pump described herein is a rotary peristaltic enteral feeding pump, the present disclosure has application to other types of peristaltic pumps (not shown), including medical infusion pumps. Additionally, one or more of the various features and aspects of the disclosure may be implemented in peristaltic pumps that use mechanisms other than rollers without departing from the scope of the present disclosure such as linear peristaltic pumps. Moreover, feeding set assemblies (not shown) that do not include cassettes may also be used within the scope of the present disclosure.

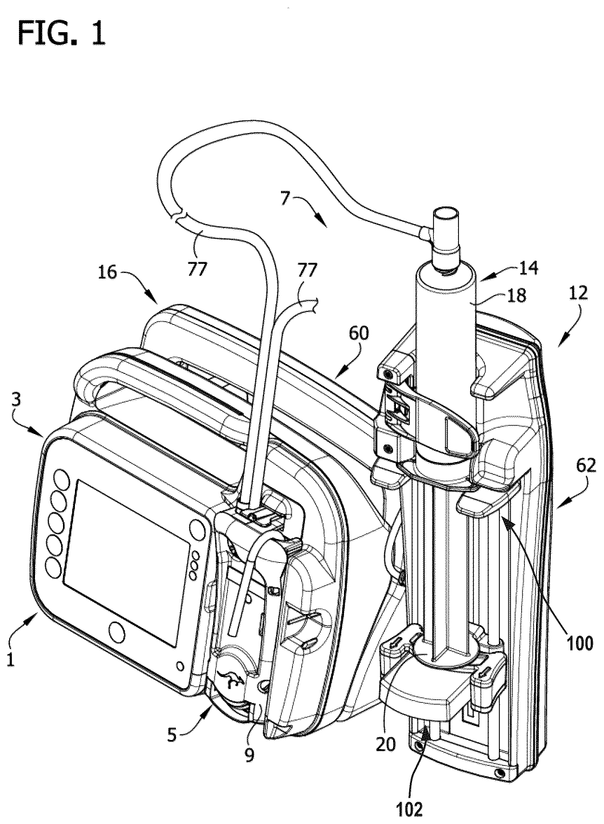

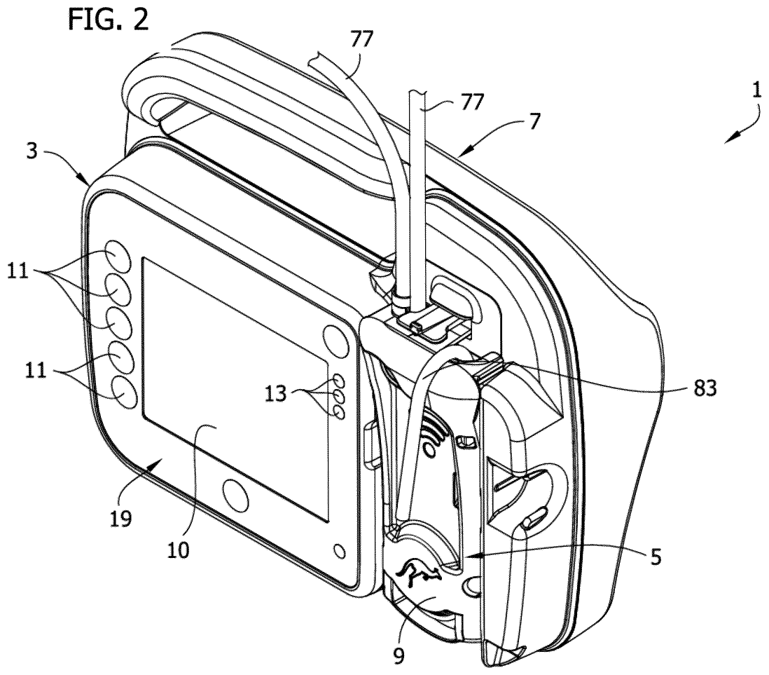

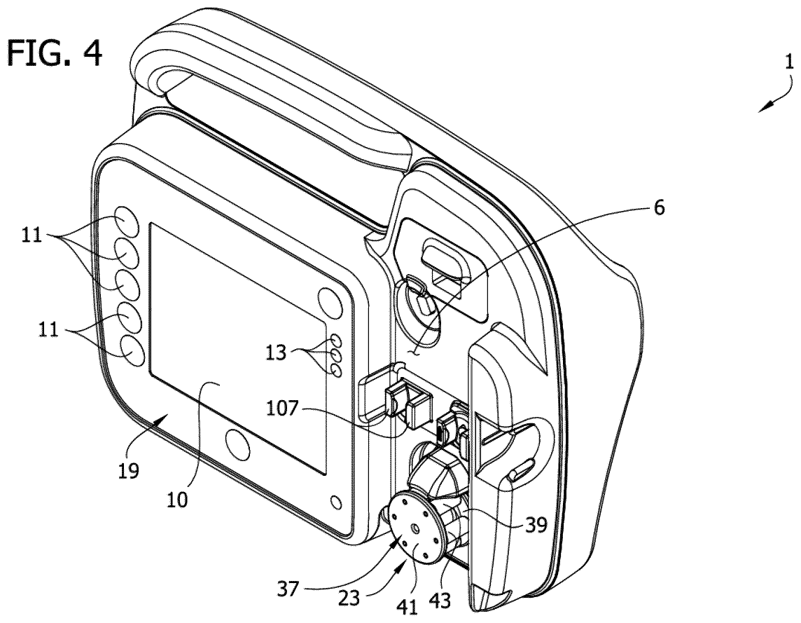

Referring now to the drawings, and in particular FIGS. 1-4, one example enteral feeding pump (broadly, “flow control apparatus”) constructed according to any one or more of the principles of the present disclosure is generally indicated at 1. The feeding pump may comprise a housing generally indicated at 3 that is constructed so as to mount a cassette, generally indicated at 5, of a feeding set assembly (broadly, a “pump set”), generally indicated at 7. The feeding set assembly 7 may include a syringe assembly 12 connected to the cassette 5 via tubing 77. The cassette 5 of the feeding set assembly 7 is releasably attachable to the housing 3. In an illustrated aspect, a cassette shell 9 of the cassette is removably received in a cassette recess 6 (FIG. 4) in the housing 3. It will be appreciated that “housing” as used herein may include many forms of supporting structures (not shown), including without limitation multi-part structures and structures that do not enclose or house the working components of the pump 1. The pump 1 has a display screen 10 on the housing 3 capable of displaying information about the status and operation of the pump. Moreover, various aspects and features of the present disclosure can be implemented without the recess 6. One or more buttons 11 which can be proximate the display screen 10 can be provided for use in controlling and obtaining information from the pump 1, and one or more light emitting diodes 13 can provide status information for the pump.

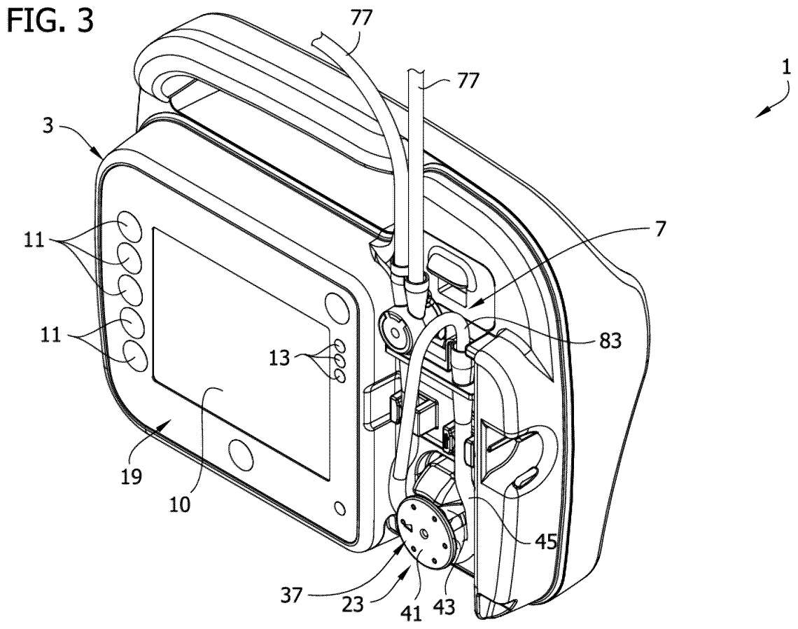

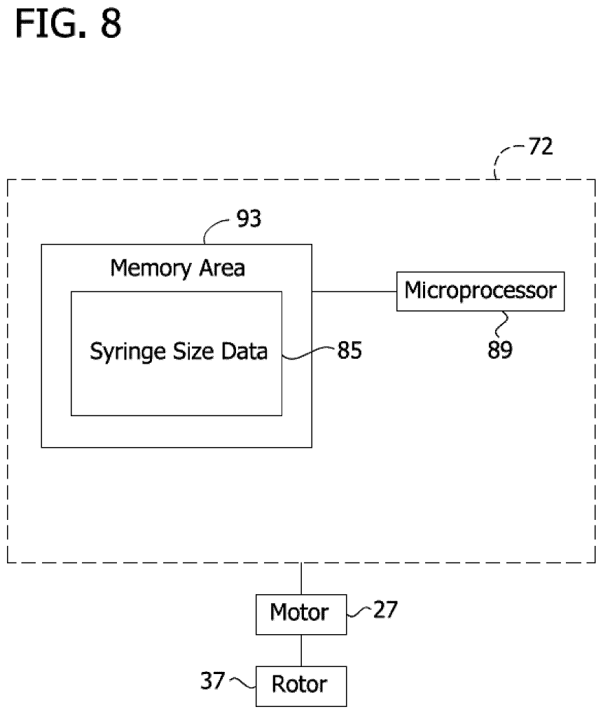

The display screen 10 may be part of a front panel (generally indicated at 19) of the housing 3 and may be removably attached to the housing. The enteral feeding pump further includes a pumping unit or device indicated generally at 23 (FIGS. 3 and 4) comprising a pump motor 27 (FIG. 8) connected to a rotor shaft (not shown). A battery (not shown) may be received in the housing 3 for powering the pump motor. A power source other than or in addition to the battery could be used to energize the pump including one or more prime motors which drive the pumping unit through the rotor shaft. The pumping unit 23 has a rotor (generally indicated at 37) which can be coupled to the rotor shaft, which may be referred to as a pumping device. The rotor 37 may include an inner disk 39, an outer disk 41, and rollers 43 mounted between the inner and outer disks for rotation relative to the disks about their longitudinal axes. The rollers 43 engage a tube 45 (FIG. 3) of the feeding set assembly 7 that forms part of the cassette 5 (which may be alternatively referred to throughout the disclosure as a pump set) pump set to deliver fluid through the feeding set assembly 7 to a subject when the cassette 5 is attached to the housing 3. For example, nutritional liquid (e.g., breast milk and/or fortifier) may be delivered to an infant using the pump 1, cassette 5, and feeding set assembly 7 based on feeding cycle. Other fluids may also be delivered using the pump 1 without departing from the scope of the disclosure. In the illustrated aspects, the fluid in the syringe 14 is drawn from the syringe by a vacuum pressure applied by the pumping unit 23. In other words, the plunger is not used to drive delivery of fluid and no pressing force is applied to a syringe 14 plunger 20. Not applying a pressing force or other force to the syringe 14 plunger 20 may improve ease of installing the syringe 14 into the holder 62 as described below an and may improve accuracy of both syringe detection and the supply of contents of the syringe 14. Not applying a pressing force may also allow for more freedom of movement using the plunger follower while not accidentally introducing movement to the syringe plunger. However, aspects of the present disclosure have equal application if the fluid from the syringe 14 is delivered from the syringe in other ways, such as by driving the plunger into the barrel of the syringe.

Referring to FIGS. 1 and 5-7, a pump support is generally indicated at 16. The pump support 16 comprises a base 60 for supporting the pump support on a horizontal support surface such as a tabletop, and a syringe holder 62 attached to the base for securing the syringe 14 to the base. It will be understood that the base could also be configured to support the pump on other surfaces or structure, including without limitation non-horizontal surfaces. As will be explained in greater detail below, the holder 62 is configured to detect the presence and/or size of the syringe 14 mounted to the holder. In one aspect, the syringe 14 and syringe holder 62 comprise the syringe assembly 12. However, the syringe 14 alone or the syringe and tubing connected to the syringe may comprise the syringe assembly. The pump support 16 supports the syringe 14 relative to the pump 1 when the pump is mounted on the pump support. Alternatively, the pump support 16 may be configured as a syringe stand such that the holder receives and supports the syringe 14 but does not also mount and/or support the pump 1. The syringe 14 may be a conventional syringe including a barrel 18, which may be graduated, and a plunger 20 slidably received in the barrel. The syringe 14 may also be of other configurations without departing from the scope of the present disclosure.

The base 60 has a flat bottom surface 64 for resting the base on a horizontal support surface. A back wall 66 extends upward from the bottom surface 64 and mounts the pump 1 to the base 60. A pair of side walls 68 extend laterally from the back wall 66 opposing opposite sides of the pump 1 when the pump is mounted to the base 60. The back wall 66 and side walls 68 together define a receiving space for the pump 1. The back wall 66 attaches to the holder 62 to locate the holder relative to the base 60.

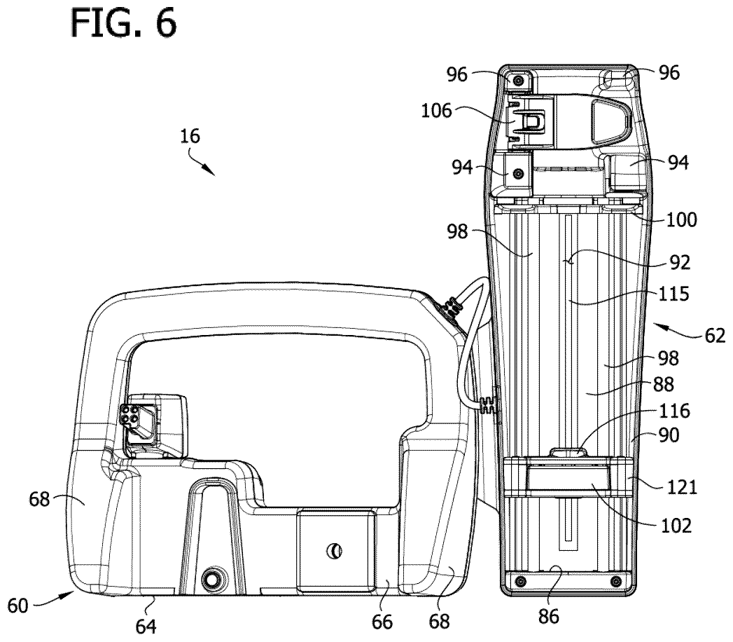

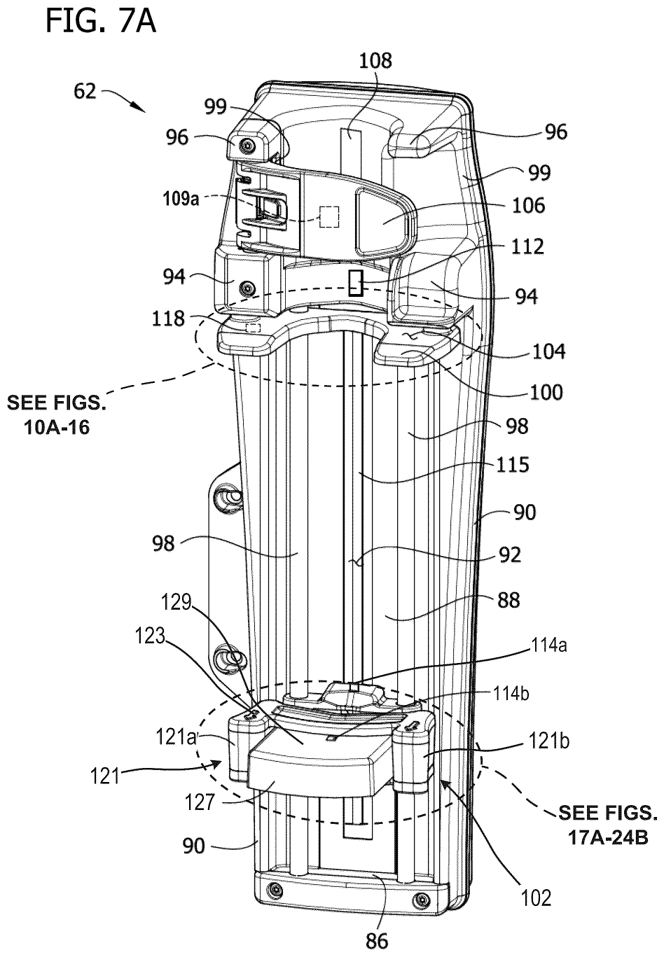

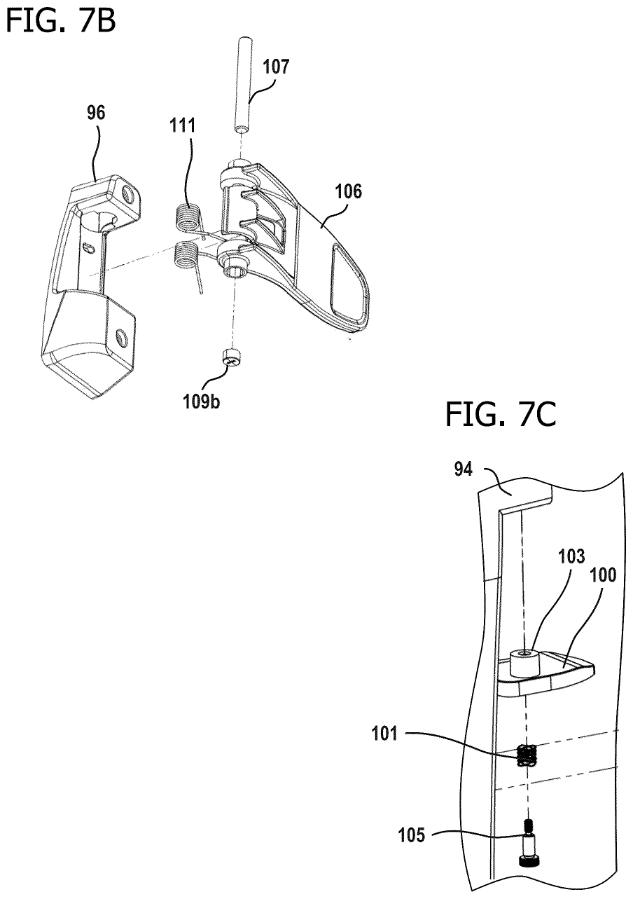

Referring to FIGS. 6 and 7, the syringe holder 62 includes a floor 86, a rear wall 88 extending from the floor, and opposing side walls 90 extending laterally from the rear wall and away from the floor. The floor 86, rear wall 88, and side walls 90 together define a receiving space 92 for at least a portion of the syringe 14. A first pair of flanges 94 extend from respective side walls 90 of the holder 62 near a top of the holder. Each side wall 90 has a recessed portion 99 above the flange 94 forming a second pair of flanges 96 longitudinally spaced upward from the first pair of flanges 94. A portion of the barrel 18 of the syringe 14 is received between the first pair of flanges 94 and between the second pair of flanges 96. The flanges 94, 96 prevent movement of the barrel 18 in the holder 62 along an axis parallel to the rear wall 88. A pair of rails or guides 98 may extend between the floor 86 and the first pair of flanges 94.

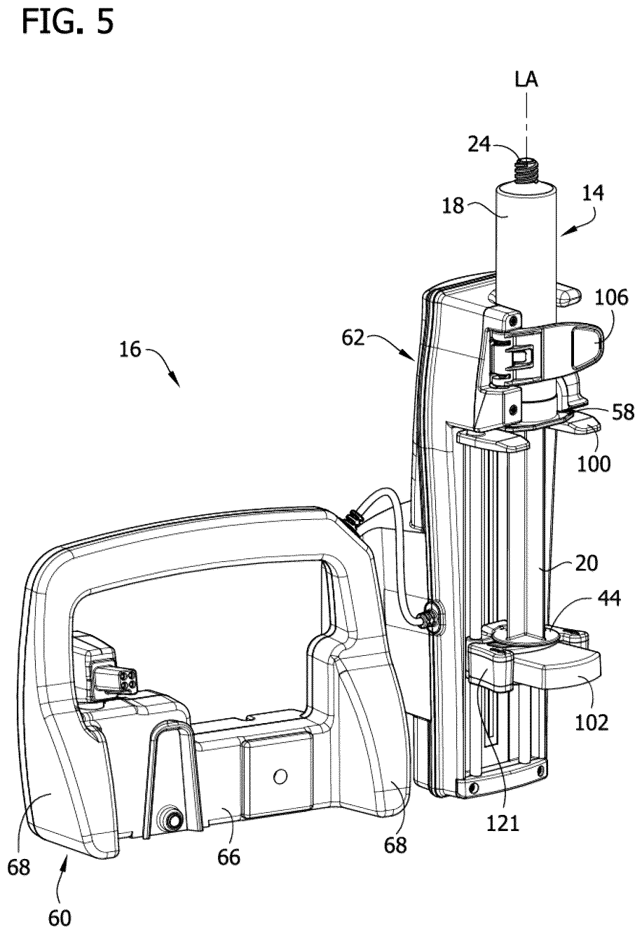

A flange holder 100 (which may be interchangeably referred to as a flange plate) is fixedly disposed at a top end of the rails 98, and a plunger follower 102 (which may alternatively be referred to as a slide plate) is configured to move or slide along the rails in response to movement of the syringe plunger 20. In some examples, the plunger follower 102 is freely slideable along the rails 98 to follow movement of the syringe plunger 20. A gap 104 is formed between the first pairs of flanges 94 and the flange holder 100. The gap 104 is configured to receive a flange 58 of the barrel 18 of the syringe 14 (FIG. 5). The gap 104 may be configured to slideably receive or otherwise engage with the flange 58 of syringe 14. In some examples, the gap 104 may have a dimension (e.g., a width or length) that is slightly smaller than a thickness of the flange 58 of the syringe 14. Thus, the flange 58 is held fixed between the flanges 94 and the flange holder 100 thereby preventing longitudinal movement of the barrel 18 of syringe 14 within the holder 62. The flanges 94 and/or the flange holder 100 may be formed of an elastic or otherwise semi-rigid or rigidly elastic material to allow either one of or both of the flanges 94 and/or the flange holder 100 to flex enough to allow for insertion of the flange 58 of syringe 14 into the gap 104, and to captively hold the flange 58 until a user or technician intentionally removes the syringe 14 from the holder 62. Further, the flange holder may be connected to the holder 62 via a spring mechanism that further allows the flange of the syringe to be installed and captively held until a user or technician intentionally removes the syringe 14 from the holder 62. FIG. 7C shows a partially expanded view of the flange holder 100. The flange holder may have one or more mounting points or portions 103 that are concavely shaped and have an opening to receive a fastener 105 therethrough and a spring or biasing member 101 therein. The fasteners 101 may pass through the center of the spring or biasing member 101 and may then be threaded into the holder 62. The partial threads on fasteners 105 and concavely shaped mounting portion may house the spring therein (held in position by the fastener 105) and allow the flange holder 100 to be biased upward while allowing movement in a downward direction when the flange of a syringe 14 is installed.

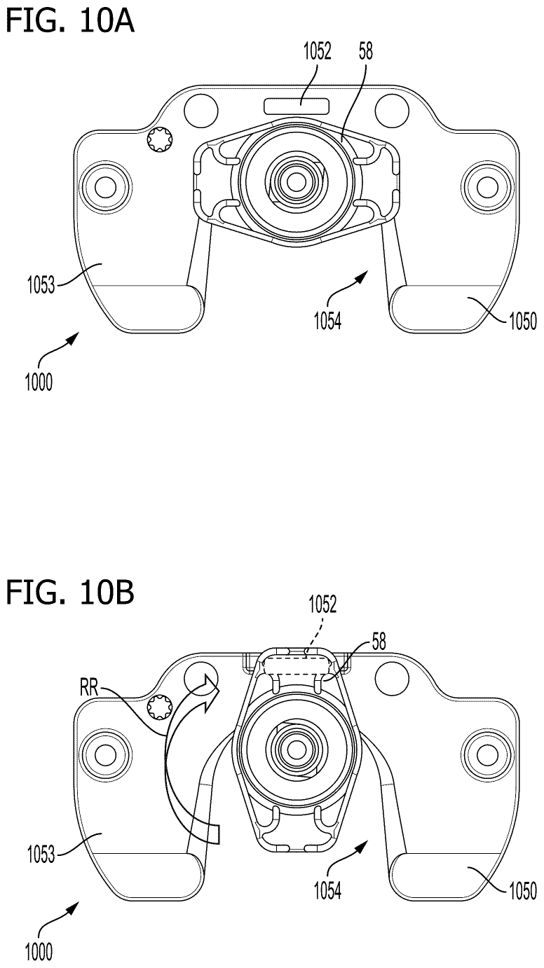

The flange holder 100 may be U-shaped and as mentioned above may be formed of a semi-flexible or rigidly elastic material such as a plastic, which may include any one or a combination of thermoplastic elastomers, monomers, polyesters copolyesters and/or equivalents thereof to name a few non-limiting examples. The flange holder 100 may be shaped and/or may include features that improve ease of installing a syringe 14, that allow for the use of multiple size syringes and/or that tactually or mechanically guide a user or technician to correctly install a syringe into the gap 104 of the holder 62. Additional details of the flange holder 100 and alternatives usable with aspects of the disclosure are described in further detail below with respect to FIGS. 10A-16.

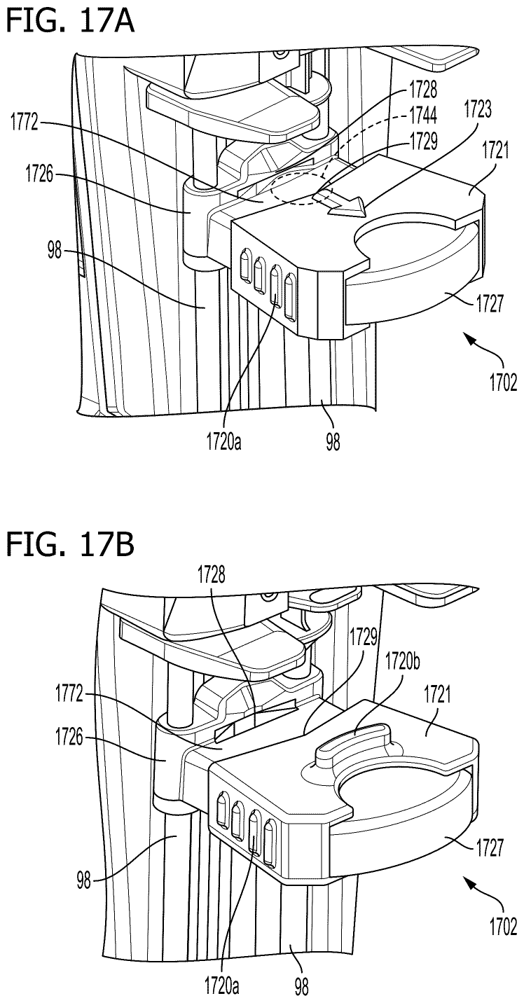

In one example, when the syringe 14 is received in the holder 62, a flange 44 of the plunger 20 may be held between a catch 121 (which may alternatively be referred to as slide) and the plunger follower 102. The catch 121 may be actuatable to slide away from the rear wall 88, for example in a direction generally indicated by arrow 123 in FIG. 4, to provide clearance for the plunger flange 44. The catch 121 can then be moved back in an opposite direction toward the rear wall 88 (i.e., in a direction generally opposing the direction of arrow 123) to secure the plunger flange 44 to the plunger follower 102. The plunger follower 102 may have one or more biasing members or springs (hidden from view) that are configured to bias the slide 121 to the resting state shown in FIG. 7A. Thus, the catch 121 is biased into an engagement position to captively engage the flange 44 of the plunger 20 so that the plunger follower 102 is removeably connected to the flange 44 (and thus follows the path of the plunger 20), for example as the pump 1 withdraws the contents of the syringe 14, until a user disengages the flange 44 from the plunger follower 102. In the example shown in FIG. 7A, to engage or otherwise install a flange 44 of the plunger 20 when installing a syringe 14 into the holder 62, a user may place their thumb or other finger(s) on a press-surface 127 of the plunger follower 102. A user may then use their index finger and middle finger or other finger(s) to pull two or more grasping portion(s) 121a and 121b toward the press-surface 127 (i.e., generally in the direction of arrow 123). The user may then place the flange 44 onto a top surface 129 of the plunger follower 102 and release or otherwise reduce the pulling force being applied to the grasping portions 121a and/or 121b (and thus allowing the catch 121 to return toward the resting position). The spring force that biases the catch 121 in a direction opposite the direction of arrow 123 may then cause a portion of the catch to captively engage the flange 44 until a user or technician intentionally removes the flange 44 by again pulling the grasping portions 121a and/or portion(s) 121b towards the press-surface 127 in a direction of arrow 123. Additional details of the plunger follower 102 and alternatives usable with aspects of the disclosure are described in further detail below with respect to FIGS. 17A-24B.

As will be explained in greater detail below, fluid being drawn from the barrel 18 of the syringe 14 causes the plunger 20 to move away from the floor 86. Because the flange 44 of the plunger 20 is captured between the catch 121 and plunger follower 102, the movement of the plunger is followed by the plunger follower 102 which moves along the rails 98 in response to movement of the plunger. A connection arm that allows for the fastening of the holder 62 onto the base 60 or pump support 16 may extend from one of the side walls 90 and is configured to attach the holder 62 to the base 60.

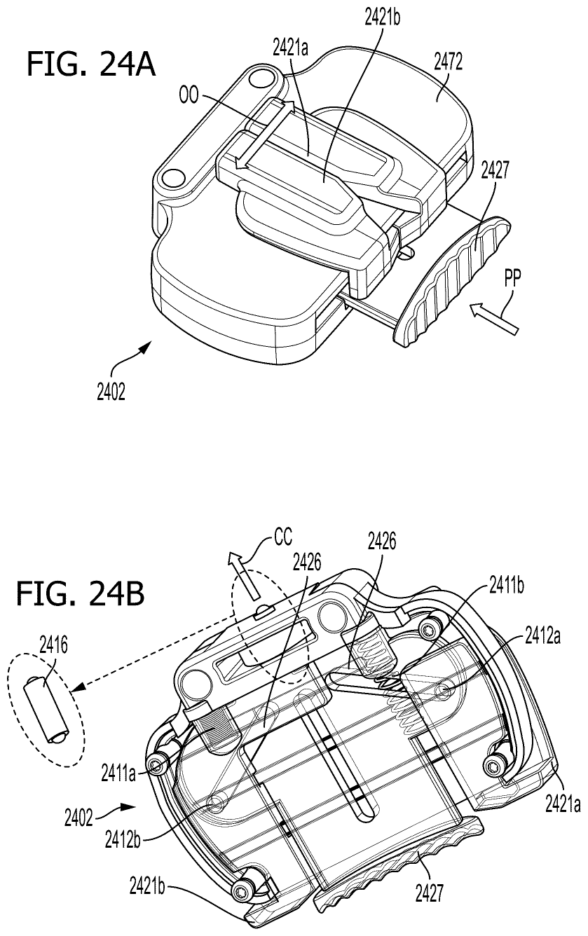

Referring to FIGS. 1 and 7, a sensor 115 may be attached to the rear wall 88 of the holder 62 to detect placement of the syringe 14 in the holder and/or the movement of the plunger 20 of the syringe relative to the holder. In the illustrated aspect, the sensor 115 comprises a linear potentiometer or may comprise a linear resistive potentiometer. However, other sensor types may be used without departing from the scope of the disclosure. For example, the sensor 115 may be a hall effect sensor, multiple hall effect sensors or an array thereof, and/or an array of hall effects devices. When the syringe 14 is properly loaded on the holder 62, a contact feature 116 on the holder (e.g., plunger follower 102) engages (i.e., comes into direct contact with) the sensor 115. In some examples, the contact feature 116 may for example be a ball detent (e.g., as shown by reference 2416 in FIG. 24B) or spring-loaded mechanical contact feature. For example, the plunger 20 may cause the contact feature 116 to come into contact with the sensor 115. In one aspect, the circuit of the senor 115 is normally closed. However, the pressure from the contact feature 116 may divide the voltage causing the analog signal to change indicating that the syringe 14 has been properly loaded onto the holder 62. If the change in the analog to digital converter (ADC) value is not detected upon loading the syringe 14 onto the holder 62, the pump 1 may continue to prompt the user to load the syringe on the holder. The sensor 115 and contact feature 116 may comprise a first syringe presence detection assembly, and a process of detecting the presence of the syringe 14 using the sensor and contact feature may comprise a first syringe presence detection routine that is operable by a controller 72 of the pump 1 operatively connected to the sensor.

Additionally, a flange sensor 112 may be associate with (e.g., disposed in or attached to) the holder 62, and a magnet 118 may be associated with (e.g., disposed in or attached to) flange holder 100.

In one aspect, the magnet 118, may for example comprise one or more permanent magnets. The aforementioned permanent magnet may for example comprise a magnetized ferromagnetic material or a material containing magnetized material(s) and/or particles. Because the flange holder 100 is cantilevered off of the rear wall 88, and the gap 104 may be slightly smaller than a thickness of the barrel flange 58, a free end of the plate is deflectable (i.e., movable downward) upon receiving the barrel flange in the gap. The downward deflection of the flange holder 100 causes movement of the magnet 118 which can be sensed by the flange sensor 112. For example, the flange sensor 112 may be hall effect sensor detecting a change in the magnetic field strength caused by the movement of the magnet 118. This change in magnetic field strength may provide a secondary indication of the presence of the syringe 14 in the holder 62. Broadly, the flange sensor 112 and magnet 118 may comprise a second syringe presence detection assembly, and a process of detecting the presence of the syringe 14 using the sensor and magnet may comprise a second syringe presence detection routine that is operable by the controller 72 of the pump 1 operatively connected to the sensor.

A door or gate 106 (broadly, a syringe clip) may be pivotably attached between one of the first pair of flanges 94 and one of the second pair of flanges 96 and moveable between an open position to allow the syringe 14 to be received in the receiving space 92, and a closed position for retaining the syringe (i.e., barrel 18) in the receiving space. FIG. 7B shows a view one example of a pivot shaft 107 that the gate 106 is configured to pivot about. One or more springs 111, may bias the gate 106 into the closed position. A sensor 108 (FIG. 7A) may be provided on the holder 62 to detect the position of the door 106 as it is moved between the open and closed positions. For example, a magnet 109a and/or 109b (FIG. 7B) may be located in the gate 106 such that a change in magnetic field is detected based on the angular position of the magnet relative to the sensor 108. Thus, the angular position of the gate 106 is detected as the gate is opened to provide a passage for the syringe barrel 18 to be received in the holder 62, and then closed around the barrel to secure the syringe to the holder. While two examples of magnet positions 109a and 109b are shown, it is noted that in one example, the magnet may be located anywhere on the gate 106. As will be explained in greater detail below, a determination of the size of the syringe 14 can be made using the angular position of the gate 106 when the barrel 18 is secured in the holder by the gate. In one aspect, the sensor 108 comprises an angular sensor. In some aspects, the angular sensor may be embedded within or otherwise located at the surface of or approximately at the surface of the rear wall 88 or a body of the holder 62. In some aspects of the disclosure, the angular sensor may be on and/or within a flexible circuit board or flexible printed circuit board. In some aspects of the disclosure, the flexible printed circuit board and/or the location of the angular sensor may allow the surfaces of the body of the holder to be easily cleaned and/or disinfected. Broadly, the sensor 108 and magnet 109 may comprise a syringe size detection assembly.

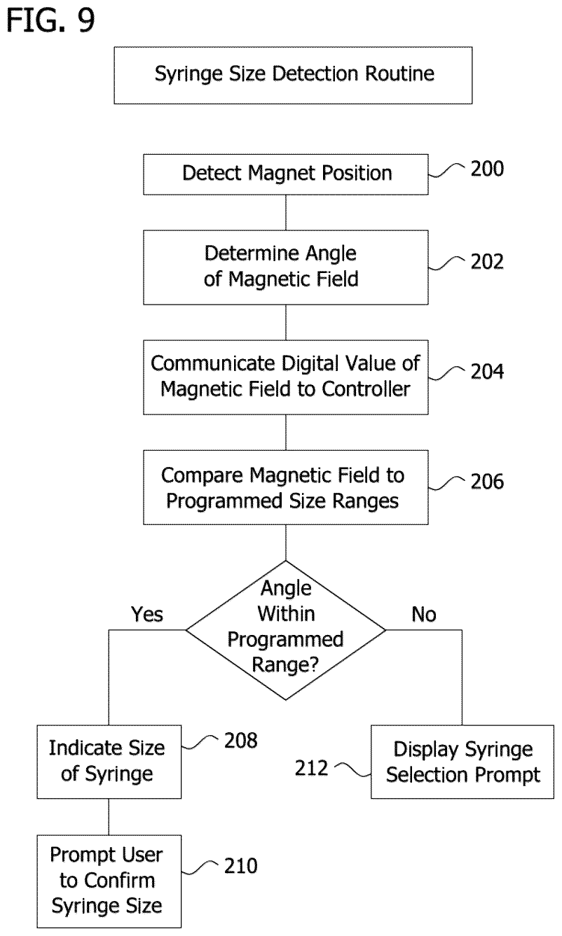

Referring to FIGS. 7A-9, a controller 72 in the pump 1 may initiate a syringe size detection routine to determine the size of the syringe received in the holder 62. The syringe size detection may be based on stored syringe size data stored in a memory 93 as described below. Once the gate 106 has fully secured the barrel 18 of the syringe 14 to the holder 62, a position of a line of demarcation between the north and south pole on the magnet 109 is detected by the sensor 108 at 200. An angle of the magnet 109 is then determined by the sensor 108 using integrated hall devices at 202. A proportional digital value of the magnetic field is then communicated to the controller 72 at 204. The magnetic field signal or syringe size reading is compared to statistically generated corresponding digital size range data 85 previously programmed and saved in a memory 93 of the controller 72 at 206. If the magnetic field angle falls within one of the programmed size ranges, and the presence of the syringe 14 was previously detected and confirmed by the two syringe presence detection routines, then the size of the syringe is indicated by the pump 1 at 208. For example, the display screen 10 of the pump 1 may show the size syringe that has been detected (e.g., 60 mL syringe). The pump 1 may then prompt the user to confirm that the detected syringe size is correct at 210. For instance, the pump 1 may display a question on the display screen 10 with the option to select “Yes” or “No” to confirm that the detect syringe size is correct. If, however, the magnetic field angle falls outside of the programmed size ranges, the pump 1 may display a prompt on the display screen 10 for the user to one of select the syringe size from a list of options or manually input the syringe size at 212. For example, the display screen 10 may show four syringe sizes and an “other” option for the user to select from. Once the syringe size has either been detected or selected by the user, the pump 1 may be operated to pump fluid from the syringe assembly. In one aspect, operation of the pump 1 to deliver fluid form the syringe 14 is prevented unless both the first and second syringe presence detection routines indicate that the syringe is present, the syringe size detection routine determines the size of the syringe, and the syringe size has been confirmed or identified.

The holder 62 may also be configured to detect movement of the plunger 20 during pumping of the fluid from the syringe 14. A contact of the potentiometer 115 may be disposed on a movable portion of the holder 62, such as the plunger follower 102, so that movement of the plunger follower 102 causes the contact to move along the potentiometer 115. Some non-limiting examples of contact(s) are shown as reference 114a and/or 114b in FIG. 7A. Because the barrel 18 is held fixed in the holder 62, as fluid is withdrawn from the barrel (e.g., via the pump 1), the plunger 20 will move into the barrel. The flange 44 of the plunger 20 is fixed to the plunger follower 102 as the plunger moves into the barrel 18 causing the plunger follower 102 to move along the rails 98. Thus, in this aspect of the disclosure, movement of the contact 114 represents the movement of the plunger 20 relative to the barrel 18 and holder 62 caused by the feeding fluid being drawn out of the syringe 14. Stated another way, the movement of the contact corresponds to the distance which the plunger 20 has advanced into the barrel 18. Further, an outer diameter of the barrel 18 can be extrapolated from the angular position that the gate 106 to identify the appropriate calibration constant to use which directly relates change in plunger 20 position to a change in fluid volume delivered. Alternatively, since the cross-sectional area of the internal cavity of the barrel 18 is known from the detection of the syringe size, the potentiometer 115 can be calibrated so that the movement of the contact 114 indicates the volume of fluid expelled from the syringe 14. In particular, by knowing the inner diameter of the barrel 18 of the syringe 14, in combination with the distance the plunger follower 102/plunger 20 has moved, the volume of fluid delivered from the syringe 14 can be determined. The potentiometer 115 may be electrically connected to the controller 72 for receiving position signals from the potentiometer 115 indicating the movement of the plunger follower 102, for example as the contents of the barrel 18 of syringe 12 are withdrawn by pump 1. The controller 72 may be located in the pump 1 or may be located remote from the pump 1 and in communication with the pump 1. For example, the controller 72 may be located in the pump support 16. In an aspect where the plunger 20 is held fixed and the barrel 18 moves relative to the plunger, the movement of the contact represents the movement of the barrel 18.

One example feeding set assembly 7 may be used for enteral feeding of neonates to achieve metered fluid delivery using the enteral feeding pump 1. In such a method, the enteral liquid is drawn into the syringe 14 by pulling back on the plunger 20. The amount of enteral liquid may be measured using graduation markings on the barrel 18 of the syringe 14. With the syringe 14 loaded in the holder 62 of the pump support 16 and attached to the tubing 77, the pump 1 is configured for delivering the feeding solution in the syringe to a subject. Operation of the pump 1 causes the rollers 43 to engage the tube 45 in the cassette shell 9 to pump the feeding solution from the syringe 14 to the subject. Engagement of the tube 45 by a roller 43 causes the rollers 43 to occlude the tube 45. If the pump support 16 is configured such that the syringe is oriented in a vertical orientation with the tip 24 facing upward, gravity does not assist in drawing feeding fluid out of the syringe. Additionally, there is no direct actuation of the plunger 20 that forces fluid upward out of the barrel 18. Thus, as the rotor 37 rotates to occlude the tube 45 with the rollers 34, air, not liquid, is first drawn out of the inlet tubing 77 and barrel 18 of the syringe 14 which increases the vacuum pressure within the syringe. After a sufficient number of rotor rotations, vacuum pressure is created in the inlet tubing 77 and syringe 14. Continued rotation of the rotor 37 will withdraw the contents of the barrel 18 (e.g., feeding fluid or product) into the inlet tubing 77 through the inlet port 69 and tubing 45 of the cassette shell 9 to be pumped by the pump 1 into the outlet tubing 83 to the subject.

The pump 1 can be programmed or otherwise controlled for operation in a desired manner. For instance, the pump 1 can begin operation to provide feeding fluid from the syringe 14 to the subject. A user such as a caregiver may select (for example) the amount of fluid to be delivered, the flow rate of the fluid, and the frequency of fluid delivery. The pump 1 may have controller 72 (FIG. 8) including a processor such as a microprocessor 89 that allows it to accept programming and/or to include pre-programmed operational routines, e.g., algorithm, that can be initiated by the user. The controller 72 may also be connected to the pump motor 27 for controlling its operation to actuate the rotor 37.

The amount of feeding fluid that is delivered to the subject is typically controlled by the number of rotations of the rotor 37 (in a counterclockwise direction as viewed in FIG. 3). In one aspect, the rotor 37 may include three rollers 43 so that each one-third of a rotation delivers one aliquot of fluid to the subject. As each roller 43 first engages the tubing 45, it pinches off the tubing thereby closing off an amount of fluid forward (i.e., toward the subject) from the fluid coming from the feeding source. The roller 43 continues in the counterclockwise rotation which pushes the pinched-off volume of fluid forward of the roller, e.g., the aliquot, toward the subject. Finally, the leading roller 43 releases engagement with the tubing 45 at about the same time the trailing roller engages the tubing for pinching it off for delivering the next aliquot of fluid. Thus, in one aspect, an algorithm may be used to determine the correct flow rate. In one aspect, when the microprocessor 89 receives a command to deliver a selected fluid flow rate, it may calculate the number of rotations within a given period of time (e.g., a feeding cycle) that will deliver a number of aliquots producing the desired flow rate. The selected flow rate may be a rate that is input or selected by the doctor, nurse or other caregiver, or may be a default feeding rate pre-programmed into the pump 1.

Aspects of the disclosure may be described in the general context of computer-executable instructions, such as program modules, executed by one or more computers or other devices. The computer-executable instructions may be organized into one or more computer-executable components or modules including, but not limited to, routines, programs, objects, components, and data structures that perform particular tasks or implement particular abstract data types. Aspects may be implemented with any number and organization of such components or modules. For example, various features or aspects are not limited to the specific computer-executable instructions or the specific components or modules illustrated in the figures and described herein. Other aspects may include different computer-executable instructions or components having more or less functionality than illustrated and described herein.

Further, the order of execution or performance of the operations in any of the aspects illustrated and described herein is not essential, unless otherwise specified. That is, the operations may be performed in any order, unless otherwise specified, and aspects may include additional or fewer operations than those disclosed herein. For example, it is contemplated that executing or performing a particular operation before, contemporaneously with, or after another operation is within the scope of one or more aspects.

In operation, microprocessor 89 of the controller 72 executes computer-executable instructions such as those illustrated in the figures to implement one or more aspects disclosed herein. Any of the various aspects may also be practiced in distributed computing environments where tasks are performed by remote processing devices that are linked through a communications network. In a distributed computing environment, program modules may be located in both local and remote computer storage media including memory storage devices.

FIGS. 10A and 10B show one example of a flange holder 1000 usable with aspects of the disclosure. The flange holder 1000 may share features with and/or may be analogous with the flange holder 100 described above with respect to FIGS. 1, 5, 6, and 7. The flange holder 1000 may be specially shaped and/or may include features that: improve ease of installing a syringe 14, that allow for the use of multiple size syringes, and/or that tactually or mechanically guide a user or technician to correctly install a syringe into the gap 104 of the holder 62. For example, the flange holder 1000 may include a tactile or guiding portion 1052 for guiding or otherwise preventing incorrect orientation of a flange 58. The example of the guiding portion 1052 in FIG. 10A is an elongated protrusion that extends from an upper surface 1053 of the flange holder 1000. The tactile or guiding portion 1052 prevents the flange 58 of a syringe from being installed incorrectly in a gap between the flange holder 1000 and a flange (e.g., flanges 94 in FIGS. 6 and 7). As shown in FIG. 10A, when the flange 58 is installed correctly, the flange clears the tactile or guiding portion 1052 of the flange holder 1000. If the flange is installed incorrectly (e.g., as shown in FIG. 10B), the flange 58 interferes with or otherwise hits the tactile or guiding portion 1052 indicating to a user that the flange 58 and/or associated syringe (e.g., syringe 14 in FIGS. 1 and 8) should be rotated (e.g., in the direction of arrow RR) to the correct orientation that clears the tactile or guiding portion 1052 (e.g., the orientation shown in FIG. 10A). The flange holder 1000 may additionally have an opening or clearance portion 1054 for clearing or for otherwise passing a plunger (e.g., plunger 20 of syringe 14 in FIGS. 1 and 5) therethrough. As shown in the example of FIGS. 10A and 10B, the clearance portion 1054 may be u-shaped. The flange holder 1000 may additionally include one or more syringe flange guide portions 1050. The syringe flange guide portions 1050 may for example be ramped or otherwise shaped to progress or ramp from a thinner profile to a thicker profile to further assist a user in installing a syringe into the gap 104 (FIG. 7A) between the flanges 94 and the flange holder (100, 1000a) of holder 62.

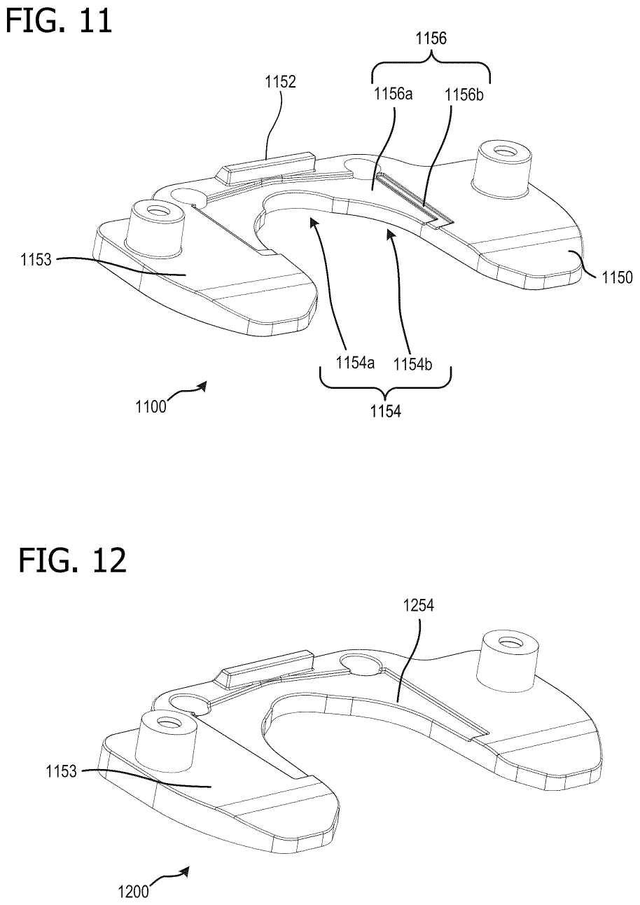

FIG. 11 shows one example of a flange holder 1100 usable with aspects of the disclosure. The flange holder 1100 may share features with and/or may be analogous with the flange holder 100 described above with respect to FIGS. 1, 5, 6, and 7. In addition to including a tactile or guiding portion 1152, which may be similar or analogous with and may provide similar advantages as tactile or guiding portion 1052 in FIG. 11, the flange holder 1100 may additionally include a surface recess 1156, which is concave or steps down from an upper surface 1153. The surface recess 1156 may additionally include a first recessed portion 1156b and a second recessed portion 1156a that that steps down from the first recessed portion 1156b. The second recessed portion 1156a and the first recessed portion 1156b may be dimensioned to receive flanges from different sizes or styles of syringes. For example, the first recessed portion 1156b may be configured to receive or otherwise be engaged with a first type of syringe flange, and the first recessed portion 1156b may be configured to receive or otherwise be engaged with a second type of syringe flange that is different from the first type of syringe flange. In one example, the first syringe flange may correspond with a syringe that has a smaller volumetric capacity than a second syringe that corresponds with the second syringe flange or vice-versa. The surface recesses described below may help with placement and detection of syringe by helping to guide the user to place the syringe's flange correctly into the recess.

The flange holder 1100 may additionally have an opening or clearance portion 1154 for clearing or for otherwise passing a plunger (e.g., plunger 20 of syringe 14 in FIGS. 1 and 5) therethrough. As shown in the example of FIG. 11, the clearance portion 1054 may have a first clearance portion 1154a with a first dimension (e.g., diameter or radius), and the clearance portion 1054 may have a second clearance portion 1154b that corresponds with a second dimension. In one example, the first clearance portion 1154a may correspond with a smaller syringe (e.g., with a smaller radius or diameter) than the second clearance portion 1154b (e.g., with a syringe with a larger radius or diameter than the smaller syringe).

Similar to the flange holder 1000 of FIGS. 10A and 10B, the flange holder 1000 may additionally include one or more syringe flange guide portions 1150. The syringe flange guide portions 1150 may for example be ramped or otherwise shaped to progress or ramp from a thinner profile to a thicker profile to further assist a user in installing a syringe into the gap 104 (FIG. 7A) between the flanges 94 and the flange holder (100, 1100) of holder 62.

FIG. 12 shows another example of a flange holder 1200 usable with aspects of the disclosure. The flange holder 1100 may share features with and/or may be analogous with the flange holder 100 described above with respect to FIGS. 1, 5, 6, and 7, the flange holder 1000 of FIGS. 10A and 10B, and/or flange holder 1100 of FIG. 11. As shown in FIG. 12, the flange holder 1200 may have a single surface recess 1254 instead of a stepped recess as described above with respect to FIG. 11. In the example shown in FIG. 12, the surface recess 1254 may extend further towards the front of the flange holder 1200 than the clearance portion 1154 of FIG. 11. The surface recess 1254 may be configured to receive or engage with multiple sizes and types of syringe flanges.

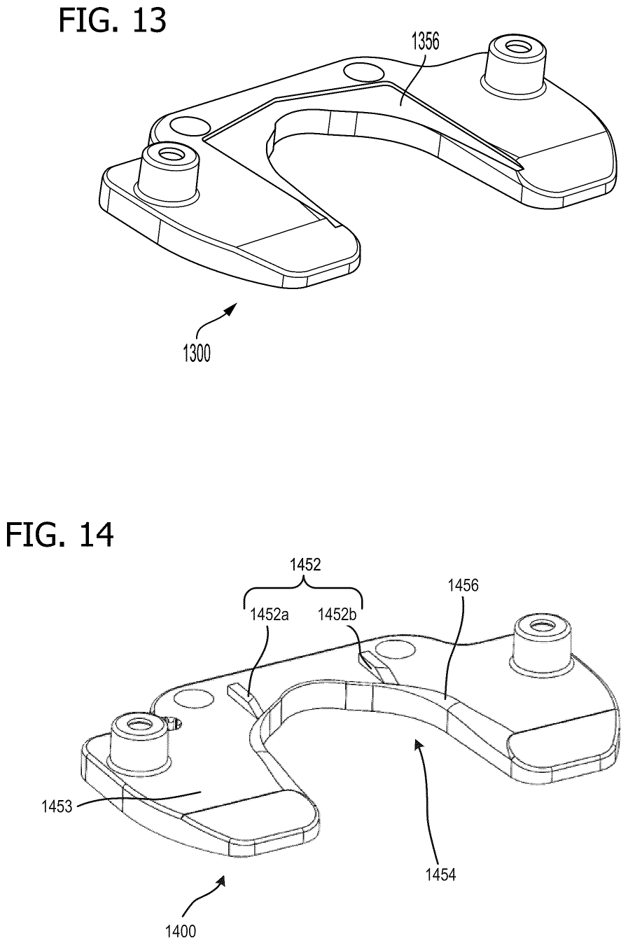

FIG. 13 shows another example of a flange holder 1300 usable with aspects of the disclosure. The flange holder 1300 may share features with and/or may be analogous with the flange holder 100 described above with respect to FIGS. 1, 5, 6, and 7, the flange holder 1000 of FIGS. 10A and 10B, the flange holder 1100 of FIG. 11, and/or the flange holder 1200 of FIG. 12. In the example shown in FIG. 13, the surface recess 1354 may extend further towards the front of the flange holder 1200 than the surface recess 1254 of FIG. 12. The surface recess 1356 may be configured to receive or engage with multiple sizes and types of syringe flanges. In the example shown in FIG. 13, the tactile or guiding portion (e.g., 1052 in FIGS. 10A and 10B, 1152 in FIG. 11 may be omitted.

FIG. 14 shows another example of a flange holder 1400 usable with aspects of the disclosure. The flange holder 1400 may share features with and/or may be analogous with the flange holder 100 described above with respect to FIGS. 1, 5, 6, and 7, the flange holder 1000 of FIGS. 10A and 10B, the flange holder 1100 of FIG. 11, the flange holder 1200 of FIG. 12, and/or the flange holder 1300 of FIG. 13. In the example shown in FIG. 14, a tactile or guiding portion 1452 may include a first protrusion 1452a and a second protrusion 1452b. The first protrusion 1452a and second protrusion 1452b may be spaced or otherwise dimensioned so that a flange (e.g., flange 58 in FIGS. 10A and 10B) fits there between or so that the flange does not fit there between depending on the desired correct orientation of the flange. The first protrusion 1452a and second protrusion 1452b may function similarly to the tactile or guiding portion 1052 in FIGS. 10A, 10B, and guiding portion 1152 in FIG. 11. Namely, the first protrusion 1452a and second protrusion 1452b may ensure correct alignment of a syringe flange with respect to the flange holder 1400. The flange holder 1400 may also include a chamfered or angled portion 1456 surrounding or partially surrounding the clearance portion 1454, which may further assist with guiding a syringe into the holder.

FIG. 15 shows a variation on an example of a flange holder 1500 usable with aspects of the disclosure. The flange holder 1500 may share features with and/or may be analogous with the flange holder 100 described above with respect to FIGS. 1, 5, 6, and 7, the flange holder 1000 of FIGS. 10A and 10B, the flange holder 1100 of FIG. 11, the flange holder 1200 of FIG. 12, the flange holder 1300 of FIG. 13 and/or the flange holder 1400 of FIG. 14. The flange holder 1500 of FIG. 15 may include a tactile or guiding portion 1552 similar to the tactile or guiding portion of FIGS. 10A, 10B, 11, and 12. Further, the flange holder 1500 may also include a chamfered or angled portion 1556 surrounding or partially surrounding the clearance portion 1554, which may further assist with guiding a syringe into the holder.

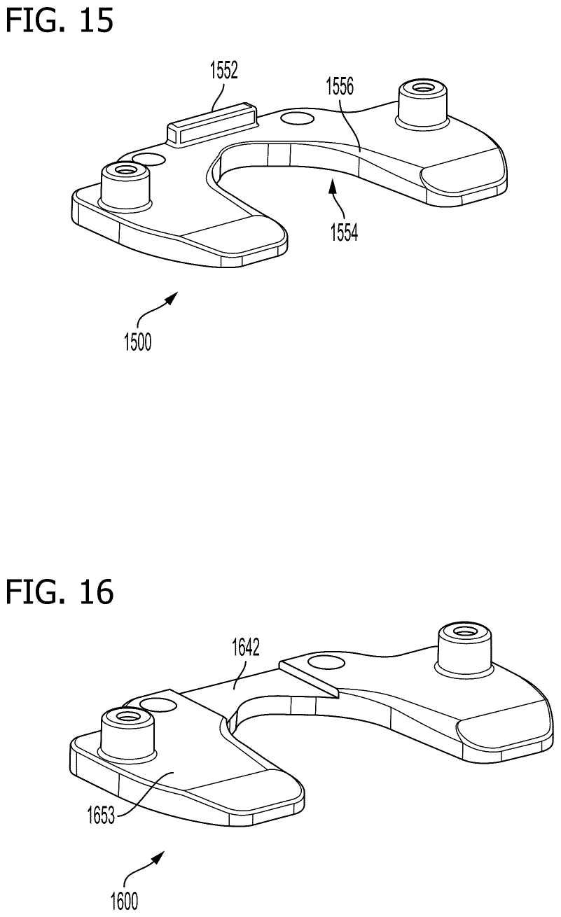

FIG. 16 shows another variation on an example of a flange holder 1600 usable with aspects of the disclosure. The flange holder 1600 may share features with and/or may be analogous with the flange holder 100 described above with respect to FIGS. 1, 5, 6, and 7, the flange holder 1000 of FIGS. 10A and 10B, the flange holder 1100 of FIG. 11, the flange holder 1200 of FIG. 12, the flange holder 1300 of FIG. 13, the flange holder 1400 of FIG. 14 an/or the flange holder 1500 of FIG. 15. The flange holder 1600 of FIG. 16 may include a tactile or guiding portion 1642 as a recess or stepped-down section with respect to a surface 1653, which may be dimensioned to receive a flange of a syringe. The tactile or guiding portion 1642 may assist a user or technician with or ensure correct alignment of a syringe flange with respect to the flange holder 1600.

FIGS. 17A and 17B show examples of followers 1702 usable with aspects of the disclosure. The followers 1702 may be analogous with or share features with the plunger follower 102 described above with respect to FIGS. 1 and 5-7. The followers 1702 may be slideably mounted on rails 98 via respective follower sliders 1726 and while not shown in FIGS. 17A and 17B may include the contact 114 and sensor aspects described above. When a syringe (e.g., syringe 14 in FIGS. 1 and 5 is installed into the holder 62, a flange (e.g., flange 44) may be removably engaged with the follower 1702 so that the follower 1702 can move with the plunger of the syringe. The dotted circle 1744 represents one example of a plunger flange (e.g., flange 44 in FIG. 5) location, when a syringe flange is removably engaged with the follower 1702. The plunger flange 1744 may be held between a catch engagement portion 1729 of a catch 1721 (which may alternatively be referred to as slide) and a second engagement portion 1728. The catch 1721 may be actuatable to slide away from the second engagement portion 1728, for example in a direction generally indicated by arrow 1723 in FIG. 17A, to provide clearance for installation of the plunger flange 1744. The catch 1721 can then be moved back in an opposite direction (i.e., opposite the direction indicated by arrow 1723 in FIG. 17A) toward the second engagement portion 1728 to secure the plunger flange 1744 by captively engaging the plunger flange within formed grooves or concavities or otherwise underneath the second engagement portion 1728 and the catch engagement portion 1729. Once the plunger flange 1744 is captively engaged between the catch engagement portion 1729 and the second engagement portion 1728, any movement of the plunger causes movement of the follower 1702. The follower 1702 may have one or more biasing members or springs (hidden from view) that are configured to bias the catch or slide 1721 to the resting state in a direction opposite arrow 1723 in FIG. 17A. Thus, the catch 1721 is biased into an engagement position to captively engage a flange of a plunger so that the plunger follower 1702 is removeably connected to the flange 1744 (and thus follows the path of the plunger) until a user disengages the flange 1744 from the plunger follower 1702. In the example shown in FIGS. 17A and 17B, to engage or otherwise install a flange 1744 of the plunger when installing a syringe into the holder 62, a user may place their thumb or other finger(s) on a press-surface 1727 of the plunger follower 1702. A user may then use their index finger and middle finger or other finger(s) to pull two or more grasping portion(s) 1720a (and/or the grasping feature hidden from view on the opposite side of the follower 1702) toward the press-surface 1727 (i.e., generally in the direction of arrow 1723). As an alternative, a user may simply pull the catch 1721 in the direction of arrow 1271 without contacting the press-surface 1727. The user may then place the flange 1744 onto a top surface 1772 of the plunger follower 1702 and release or otherwise reduce the pulling force being applied to the grasping portion(s) 1720a, thus allowing the catch 1721 to return toward the resting position due to the biasing force of the aforementioned biasing member or spring. The biasing/spring force that biases the catch 1721 in a direction opposite the direction of arrow 1723 may then cause the catch engagement portion 1729 and the second engagement portion 1728 to captively engage the flange 1744 until a user or technician intentionally removes the flange 1744 by again pulling the grasping portion(s) 1720a towards the press-surface 1727 in a direction of arrow 1723. FIG. 17b shows a variation of the catch 1721 with a protrusion 1720b, which may further assist a user or technician with installation and removal of the flange 1744 when installing or removing a syringe. While hidden from view in FIGS. 17A and 17B, the follower 1702 (and additional variations described below) may include the contact feature 116 described above that engages (i.e., comes into direct contact with) the sensor 115. In some examples, the contact feature 116 may for example be a ball detent (e.g., as shown by reference 2416 in FIG. 24B) or spring-loaded mechanical contact feature.

FIG. 18 shows a follower 1802 according to aspects of the disclosure. The follower 1802 may share features with or may be analogous with the plunger follower 102 described above with respect to FIGS. 1 and 5-7 and/or followers 1702 of FIG. 17A and 17B. In the variation shown in FIG. 18, the catch 1821 has a downward protruding grasping portion, which may further assist a user with installation and/or removal of a plunger flange.

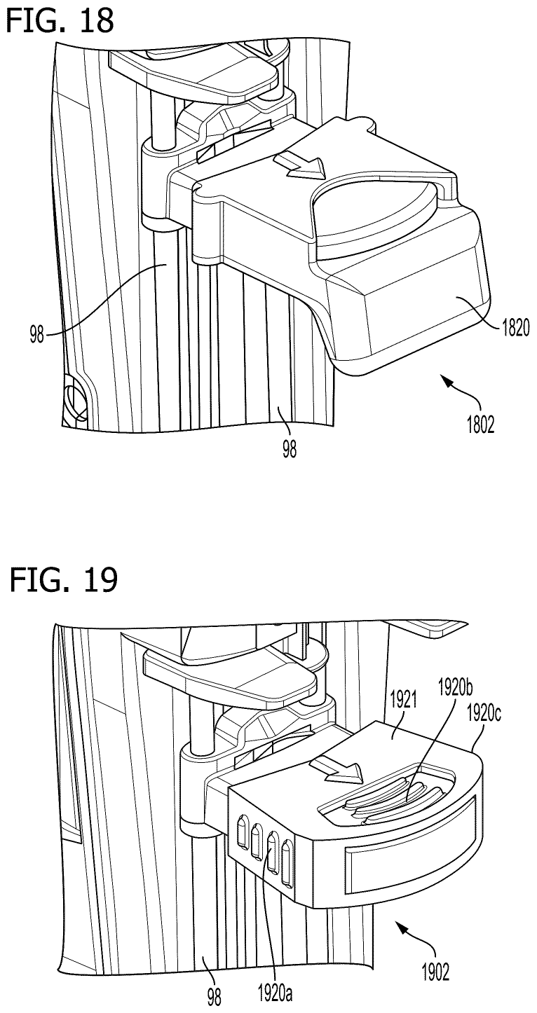

FIG. 19 shows a follower 1902 according to aspects of the disclosure. The follower 1902 may share features with or may be analogous with the plunger follower 102 described above with respect to FIGS. 1 and 5-7, followers 1702 of FIGS. 17A and 17B and/or follower 1802 in FIG. 18. In the variation shown in FIG. 19, the catch 1921 has a semi-circular front and multiple grasping portions 1920a, 1920b, and 1920c, with protrusions and/or concavities with protrusions therein which may further assist a user with installation and/or removal of a plunger flange.

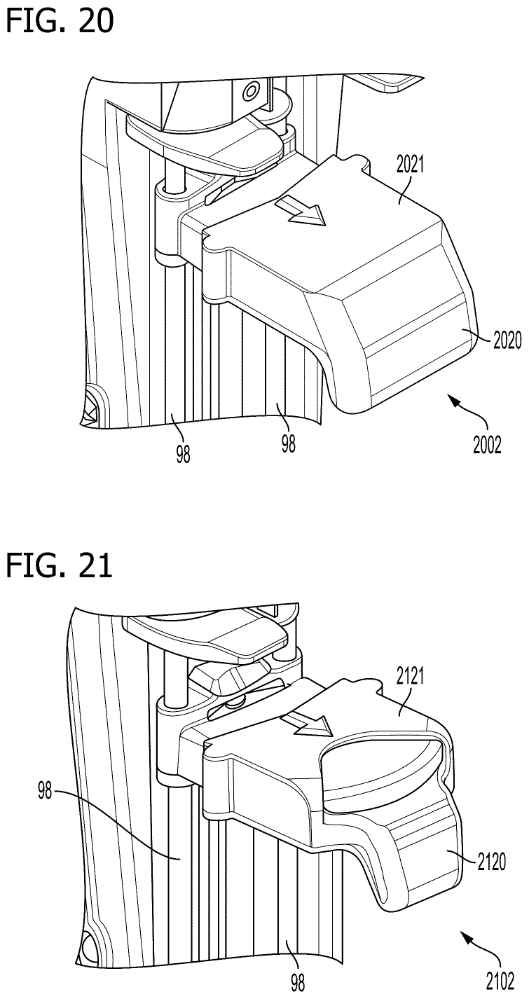

FIGS. 20 and 21 show additional examples of followers according to aspects of the disclosure. Followers 2002 and 2102 may share features with or may be analogous with the plunger follower 102 described above with respect to FIGS. 1 and 5-7, followers 1702 of FIGS. 17A and 17B follower 1802 in FIG. 18, and/or follower 1902 in FIG. 19. In the variation shown in FIGS. 20 and 21, the catches 2021 and 2121 have downward protruding grasping portions 2020 and 2120, respectively, which may further assist a user with installation and/or removal of a plunger flange.

FIG. 22 shows an example of a follower according to aspects of the disclosure. The follower 2202 may share features with or may be analogous with the plunger follower 102 described above with respect to FIGS. 1 and 5-7, followers 1702 of FIGS. 17A and 17B follower 1802 in FIG. 18, follower 1902 in FIG. 19, follower 2002 in FIG. 20 and/or follower 2102 in FIG. 21. FIG. 22 shows an example of the follower sliders 2226 with openings for receiving the rails 98 therethrough.

FIG. 22 shows one example of a concavity or groove 2228a of the second engagement portion 2228 that is configured to receive and engage with the flange (e.g., flange 44 in FIG. 5 and 1744 in FIG. 17) of the plunger when a user pulls the two or more grasping portion 2220a and 2220b toward the press-surface 2027 (i.e., generally in the direction of the arrows embossed in the catch 2221). FIG. 22 shows an alternative example of a slideable catch 2221 and grasping portions 2220a and 2220b that are usable in any combination with the aspects described herein.

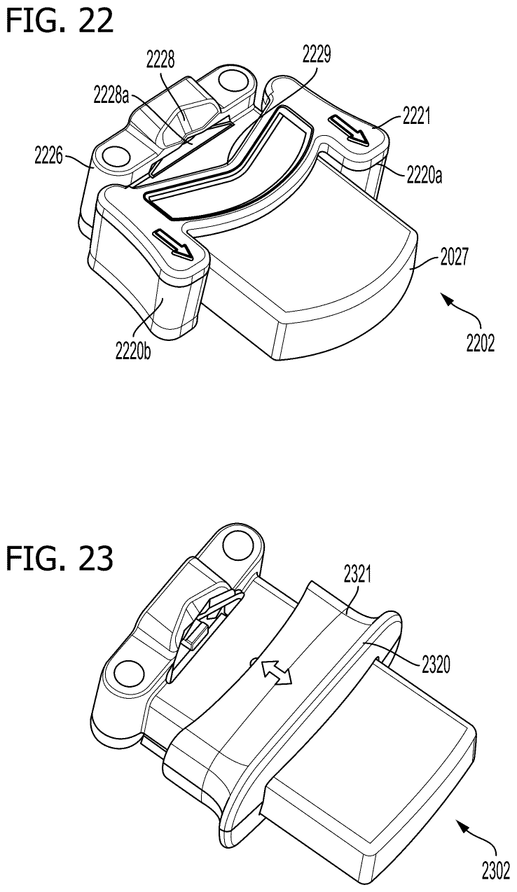

FIG. 23 an example of a follower according to aspects of the disclosure. The variation of follower 2302 shown in FIG. 23 has a catch 2321 that has a grasping feature 2320 that is shaped as an annular protruding lip that may improve a user's ability to install or remove a syringe. The follower 2302 of FIG. 23 may share features with or may be analogous with the plunger follower 102 described above with respect to FIGS. 1 and 5-7, followers 1702 of FIGS. 17A and 17B follower 1802 in FIG. 18, follower 1902 in FIG. 19, follower 2002 in FIG. 20, follower 2102 in FIG. 21 and/or follower 2201 in FIG. 22.

FIGS. 24A and 24B show a top view and a partial see-through bottom view of a follower according to aspects of the disclosure. The follower 2402 may share features with or may be analogous with the plunger follower 102 described above with respect to FIGS. 1 and 5-7, followers 1702 of FIGS. 17A and 17B follower 1802 in FIG. 18, follower 1902 in FIG. 19, follower 2002 in FIG. 20, follower 2102 in FIG. 21, follower 2201 in FIG. 22, and/or follower 2302 in FIG. 23. In one aspect, the example follower 2402 may differ from the aforementioned followers in that a user may press a release portion 2427 (e.g., in direction PP) to open a first catch 2421a and a second catch 2421b generally in the direction indicated by arrow OO. Once a user presses the release portion 2427 a user may place a plunger flange (e.g., a flange 44 as shown in FIG. 5) onto surface 2472 and release the release portion 2427 thus causing the first catch 2421a and second catch 2421b to close again (i.e., move in directions generally opposite arrows OO) onto the plunger flange and captively engage with flange. As with the aspects described above, the follower 2402 according to this aspect allows for a variety of syringe sizes (and thus flange sizes) to be installed into the holder 62 (FIGS. 1 and 5-7).

In one aspect, the first catch 2421a and/or the second catch 2421b may be biased toward one another via a biasing member or member(s) 2411a and/or 2411b. In one aspect, the biasing members 2411a and/or 2411b may tension the first catch 2421a and/or the second catch 2421b toward one another until a user presses the release portion 2427 to overcome the biasing force of the biasing members 2411a and/or 2411b. In one example, the biasing members 2411a and/or 2411b may be a push or pull spring. The release portion 2427 may for example have one or more internal tracks 2426 that are configured to engage with followers 2412a and/or 2412b corresponding to each of the first catch 2421a and/or the second catch 2421b. Pressing the release portion 2427 in direction PP with enough force to overcome the biasing force of the biasing members causes the followers 2412a and/or 2412b to travel along the one or more internal tracks 2426 which causes the first catch 2421a and second catch 2421b to separate, allowing a syringe flange to either be installed into or removed from the follower 2402.

As further shown in FIG. 24B, the follower may additionally include a contact feature 2416, which may indicate when a syringe 14 is properly loaded on the holder 62 and/or loaded into the follower. In the example shown in FIG. 24b, the contact feature 2416 may be a ball detent or a contact feature that moves in direction CC when a syringe (e.g., plunger flange) is installed into the follower. In some examples, the plunger flange may directly contact and apply a force in direction CC when installed in the follower. In other examples, the release of tension change in spacing (e.g., an increased distance between either the first catch and second catch and/or the catch engagement portion and second engagement portion associated with a plunger being installed) may progress the contact feature 2416 in direction CC. As described above, in some examples, the contact feature coming into contact with the sensor 115 indicates the proper installation of a syringe. In one example, the circuit of the sensor 115 is normally closed. However, the pressure from the contact feature 2416 may divide the voltage causing the analog signal to change indicating that the syringe 14 (FIG. 5) has been properly loaded onto the holder 62. If the change in the analog to digital converter (ADC) value is not detected upon loading the syringe 14 onto the holder 62, the pump 1 may continue to prompt the user to load the syringe on the holder. It is noted that while the contact feature 2416 is hidden from view in FIGS. 17A-23, the contact feature is applicable to and may be included in the features described above with respect to FIGS. 17A-23.

When introducing elements of the present disclosure or the aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

Throughout the present disclosure, the terms “substantially” or “approximately” may be used as a modifier for a geometric relationship between elements or for the shape of an element or component. While the terms substantially or approximately are not limited to a specific variation and may cover any variation that is understood by one of ordinary skill in the art to be an acceptable variation, some examples are provided as follows. In one example, the term substantially or approximately may include a variation of less than 10% of the dimension of the object or component. In another example, the term substantially or approximately may include a variation of less than 5% of the object or component. If the term substantially or approximately is used to define the angular relationship of one element to another element, one non-limiting example of the term substantially or approximately may include a variation of 5 degrees or less. These examples are not intended to be limiting and may be increased or decreased based on the understanding of acceptable limits to one of skill in the relevant art.

For purposes of the present disclosure, directional terms are expressed generally with relation to a standard frame of reference when the system and apparatus described herein is installed in an in-use orientation. Further, in order to provide context to the current disclosure, a broad overview of the discovered deficiencies of various systems and an example implementation of the current disclosure and the advantages provided by the disclosure are described below. Further details of example implementations of the current disclosure are described in detail with reference to the figures below.

The terms “first,” “second,” “third,” and “fourth,” among other numeric values, may be used in the present disclosure. It will be understood that, unless otherwise noted, those terms are used in their relative sense only. In particular, in some aspects, certain components may be present in interchangeable and/or identical multiples (e.g., pairs). For these components, the designation of first, second, third, and/or fourth may be applied to the components merely as a matter of convenience in the description of one or more of the aspects of the disclosure.

In view of the above, it will be seen that the several objects of the disclosure are achieved and other advantageous results attained.

As various changes could be made in the above constructions without departing from the scope of the disclosure, it is intended that all matter contained in the above description and shown in the accompanying drawings shall be interpreted as illustrative and not in a limiting sense.

What is claimed is:

1. A flow control apparatus for use with a pump set to deliver fluid from a feeding source through the pump set to a subject, the flow control apparatus comprising:
a pumping device capable of acting on the pump set to produce a fluid flow within the pump set during a feeding cycle; and
a controller in communication with the pumping device for controlling operation of the pumping device to producing a flow of fluid in the pump set, the controller including a processor and a memory, the controller being adapted to store in the memory, syringe size data, the controller configured to execute in the processor a syringe detection program to determine a presence and size of the syringe loaded onto the flow control apparatus, the syringe detection program comprising a first syringe presence detection routine to indicate the presence of the syringe, a second syringe presence detection routine to confirm the presence of the syringe, and a syringe size detection routine where a syringe size reading is compared to the syringe size data to determine the size of the syringe.

2. The flow control apparatus of claim 1, wherein operation of the pumping device is prevented until the first and second syringe presence detection routines indicate that the syringe is present, and the syringe size detection routine identifies the size of the syringe.

3. The flow control apparatus of claim 1, wherein the first syringe presence detection routine comprises detecting the presence of a plunger of the syringe.

4. The flow control apparatus of claim 3, further comprising a sensor and a ball detent configured to contact the sensor when the syringe is loaded on the flow control apparatus.

5. The flow control apparatus of claim 4, wherein a change in an analog to digital converter signal sensed by the sensor indicates the presence of the plunger of the syringe.

6. The flow control apparatus of claim 5, wherein the sensor is one of a linear potentiometer or an array of hall effect devices.

7. The flow control apparatus of claim 3, wherein the second syringe presence detection routine comprises detecting the presence of a barrel of the syringe.

8. The flow control apparatus of claim 7, further comprising a sensor and a magnet configured to indicate the presence of the barrel of the syringe.

9. The flow control apparatus of claim 8, wherein a change in a magnetic field strength sensed by the sensor indicates the presence of the barrel of the syringe.

10. The flow control apparatus of claim 7, wherein the syringe size detection routine comprises detecting an angle of a magnetic field generated by a magnet on the flow control apparatus.

11. A syringe detection device for use in a flow control apparatus for delivering fluid from a syringe to a subject, the device comprising:
a syringe holder for securing the syringe to the flow control apparatus, the syringe holder comprising a body for receiving at least a portion of the syringe and a clip pivotally attached to the body for retaining a barrel of the syringe to the syringe holder;
a magnet at the clip; and
an angular sensor attached to the body and configured to detect an angle of a magnetic field generated by the magnet, the angle of the magnetic field indicating a size of the syringe when the clip is pivoted to retain the barrel of the syringe to the syringe holder.

12. The device of claim 11, wherein the magnet is imbedded in the clip and the angular sensor is embedded in the body.

13. The device of claim 12, wherein the angular sensor is located on a flexible printed circuit board.

14. The device of claim 11, wherein the magnet comprises a permanent magnet.

15. A syringe detection device usable with a flow control apparatus for delivering fluid to a subject, the device comprising:
a syringe holder for removeably securing a syringe thereto, the syringe holder comprising a body for receiving at least a portion of the syringe and a flange plate movably attached to the body and configured to engage a flange of the syringe when the syringe is retained to the holder;
a magnet attached to the flange plate; and
a sensor attached to the body and configured to detect a change in a magnetic field generated by movement of the magnet as a result of the movement of the flange plate when the flange of the syringe engages the flange plate, the change in the magnetic field indicating the presence of the syringe in the holder.

16. The device of claim 15, wherein the flange plate is cantilevered from the body to facilitate movement of the flange plate relative to the body.

17. The device of claim 15, wherein the sensor is a hall effect sensor.

18. The device of claim 15, wherein the syringe holder further comprises a follower that is configured to move with a plunger of the syringe as a content of the syringe is withdrawn therefrom, wherein the follower further comprises a moveable catch configured to captively engage a plunger of the syringe when the syringe is retained to the holder.

19. The device of claim 18, wherein the follower is configured to slideably move along a rail of the syringe holder as the content of the syringe is withdrawn.

20. A method of detecting a syringe loaded on a flow control apparatus, the method comprising:
performing a first syringe presence detection routine to detect the presence of the syringe on the flow control apparatus;
performing a second syringe presence detection routine to confirm the presence of the syringe on the flow control apparatus; and
performing a syringe size detection routine to determine a size of the syringe.

21. The method of claim 20, further comprising operating the flow control apparatus to deliver fluid through a pump set on the flow control apparatus only after the first and second syringe presence detection routines indicate that the syringe is present, and the syringe size detection routine identifies the size of the syringe.

22. The method of claim 20, wherein the first syringe presence detection routine detects the presence of a plunger of the syringe, and the second syringe presence detection routine detects the presence of a barrel of the syringe and a third syringe detection routine confirms the presence of a syringe flange.
